(19) United States
(12) Patent Application Publication
(10) Pub. No.: US 2009/0187058 A1
(43) Pub. Date: Jul. 23, 2009

(54) PROCESS FOR THE PREPARATION OF AN OLEFIN

(76) Inventors: Leslie Andrew Chewter, Amsterdam (NL); Michiel Johannes Franciscus Maria Verhaak, Amsterdam (NL); Jeroen Van Westrenen, Amsterdam (NL)

Correspondence Address: SHELL OIL COMPANY, P O BOX 2463, HOUSTON, TX, 772522463, US

(21) Appl. No.: 12/301,134

(22) PCT Filed: May 16, 2007
(86) PCT No.: PCT/EP07/54752

§ 371 Date: Dec. 8, 2008

(30) Foreign Application Priority Data
May 19, 2006 (EP) 06114278.2

(51) Int. Cl. C07C 2/86

(52) U.S. Cl. 585/639

(57) ABSTRACT

Process for the preparation of an olefin comprising a) reacting a combination of an oxygenate feed selected from the group consisting of alcohols and ethers, and an olefinic co-feed in a reactor in the presence of a one-dimensional zeolite having 10-membered ring channels to prepare an olefinic reaction mixture; b) separating the olefinic reaction mixture into at least a first olefinic product fraction and a second olefinic fraction containing olefins with 4 or more carbon atoms; and c) recycling at least part of the second olefinic fraction obtained in step b) to step a) as olefinic co-feed.

### FIELD OF THE INVENTION

This invention relates to a process for the preparation of an olefin, such as ethene and/or propene. In specific this invention relates to a process for the conversion of oxygenates into olefins.

### BACKGROUND OF THE INVENTION

Processes for the preparation of olefins are known in the art.

U.S. Pat. No. 6,797,851 describes a process for making ethene and propene from an oxygenate feed using two or more zeolite catalysts.

In a first stage, an oxygenate feed is contacted with a first zeolite catalyst containing ZSM-5. The resulting conversion product contains an olefins composition. The olefin composition from the conversion reaction, with or without prior separation of ethylene and propylene, is then contacted with another zeolite catalyst in a second stage. The catalyst is such second stage is a one-dimensional zeolite having 10-membered ring channels, including ZSM-22, ZSM-23, ZSM-35, ZSM-42 or mixtures thereof. The eventual product comprises ethene, propene and C₄₊ olefins. The C₄₊ olefins may be partly recycled to the first reaction as olefinic co-feed of the oxygenate feed.

In the only example, pure methanol is converted by a two-step process into several olefins.

It would be desirable to have an improved process, in which olefins, such as ethene and/or propene, can be prepared from oxygenates in just one step with just one zeolite with a sufficient conversion rate.

### SUMMARY OF THE INVENTION

It has now been found that when a one-dimensional 10-membered ring is used for the oxygenate conversion step and part of the product of the such oxygenate conversion step is recycled as an olefinic co-feed, a first step as described in U.S. Pat. No. 6,797,851 is no longer necessary and a process for the preparation of an olefin, such as ethene and/or propene, can be carried out with just one zeolite in just one step with a sufficient conversion rate.

Accordingly the present invention provides a process for the preparation of an olefin comprising
a) reacting a combination of an oxygenate feed selected from the group consisting of alcohols and ethers, and an olefinic co-feed in a reactor in the presence of a one-dimensional zeolite having 10-membered ring channels to prepare an olefinic reaction mixture;
b) separating the olefinic reaction mixture into at least a first olefinic product fraction and a second olefinic fraction containing olefins with 4 or more carbon atoms; and
c) recycling at least part of the second olefinic fraction obtained in step b) to step a) as olefinic co-feed.

The process of the present invention enables the use of one single zeolite catalyst. Further, it enables the conversion of the oxygenate feed to olefins over the above-mentioned zeolite which may be cumbersome. Such is illustrated in table 2B of EP-A 0485145 where it is shown that one-dimensional zeolites having 10-membered ring channels, such as zeolites of the TON-type, are not capable of converting an oxygenate at a reasonable rate in the absence of any olefin. Moreover, the process of the present invention provides for excellent propene selectivity.

### DETAILED DESCRIPTION OF THE INVENTION

By an olefinic co-feed is understood a feed containing one or more olefins. The olefinic co-feed can contain one olefin or a mixture of olefins. Suitably the olefinic co-feed contains a mixture of olefins. Apart from olefins, the olefinic co-feed may contain other hydrocarbon compounds, such as for example paraffinic, alkylaromatic, aromatic compounds or a mixture thereof. Preferably the olefinic co-feed comprises more than 50 wt %, more preferably more than 80 wt %, still more preferably more than 90 wt % and most preferably in the range from 95 to 100 wt % of olefin(s). An especially preferred olefinic co-feed consists essentially of olefin(s).

Any non-olefinic compounds in the olefinic co-feed are preferably paraffinic compounds. If the olefinic co-feed contains any non-olefinic hydrocarbon, these are preferably paraffinic compounds. Such paraffinic compounds are preferably present in an amount in the range from 0 to 10 wt %, more preferably in the range from 0 to 5 wt %, still more preferably in the range from 0 to 1 wt % and most preferably in an amount of less than 0.5 wt %.

By an olefin is understood an organic compound containing at least two carbon atoms connected by a double bond. A wide range of olefins can be used. The olefin can be a mono-olefin, having one double bond, or a poly-olefin, having two or more double bonds. Preferably olefins present in the olefinic co-feed are mono-olefins.

The olefin(s) can be a linear, branched or cyclic olefin. Preferably, olefins present in the olefinic co-feed are linear or branched olefins.

Preferred olefins have in the range from 2 to 12, preferably in the range from 3 to 10, and more preferably in the range from 4 to 8 carbon atoms.

Examples of suitable olefins that may be contained in the olefinic co-feed include 1-butene, 2-butene, iso-butene (2-methyl-1-propene), 1-pentene, 2-pentene, 2-methyl-1-butene, 2-methyl-2-butene, 3-methyl-1-butene, 3-methyl-2-butene, 1-hexene, 2-hexene, 3-hexene, 2-methyl-1-pentene, 2-methyl-2-pentene, 3-methyl-1-pentene, 3-methyl-2-pentene, 4-methyl-1-pentene, 4-methyl-2-pentene, 2,3-dimethyl-1-butene, 2, 3-dimethyl-2-butene, 3,3-dimethyl-1-butene, cyclopentene, methylcyclopentene or cyclohexene, heptenes, octenes, nonenes and decenes. The preference for specific olefins in the olefinic co-feed may depend on the purpose of the process.

In one embodiment, where the purpose of the process is to prepare mainly propene, the olefinic co-feed preferably contains olefins having 4 or more carbon atoms (i.e. C₄+ olefins), such as butenes, pentenes, hexenes and heptenes.

In another embodiment, where the purpose of the process is to prepare ethene, propene, pentene and/or hexene, the olefinic co-feed preferably contains only olefins having 4 carbon atoms.

Of the above embodiments, the first embodiment wherein mainly propene is prepared and the olefinic co-feed contains olefins having 4 or more carbon atoms (i.e. C₄+ olefins), such as butenes, pentenes, hexenes and heptenes, is preferred.

The olefinic co-feed can comprise hydrocarbons, preferably olefins, provided by the recycle of step c) and hydrocarbons, preferably olefins, obtained from some other source. For example part of the olefinic co-feed may be derived from a steam cracker or catalytic cracker, for example a stream comprising butenes and/or butanes.

The olefinic co-feed in step a) preferably consists for at least 50 wt %, more preferably at least 80 wt %, still more preferably from 90 to 100 wt % of recycled olefins from step c). Most preferably the olefinic co-feed consists essentially of the recycled part of the second olefinic fraction obtained in step c).

By oxygenate in this specification is understood a compound selected from the group consisting of alcohols and ethers. Suitably, the oxygenate comprises at least one oxygen-bonded alkyl group. The oxygen-bonded alkyl group preferably comprises 1 to 4 carbon atoms, more preferably 1 or 2 carbon atoms and most preferably one carbon atom. The oxygenate can comprise one or more of such oxygen-bonded C₁-C₄ alkyl groups. Preferably the oxygenate comprises one or two oxygen-bonded C₁-C₄ alkyl groups. Examples of preferred oxygenates include alkanols, such as methanol, ethanol, isopropanol, ethylene glycol, propylene glycol; and dialkyl ethers, such as dimethylether, diethylether, methylethylether. Cyclic ethers such as tetrahydrofuran and dioxane, are also suitable.

Preferably the oxygenate is chosen from the group of alkanols and dialkyl ethers consisting of dimethylether, diethylether, methylethylether, methanol, ethanol and isopropanol.

More preferably an oxygenate is used having at least one oxygen-bonded C₁ or C₂ alkyl group, still more preferably at least one oxygen-bonded C₁ group. Most preferably the oxygenate is methanol or dimethylether.

In a preferred embodiment, where the oxygenate is methanol, such methanol is obtained from natural gas. For example by a process as described in Industrial Organic Chemistry 3rd edition page 28.

In another preferred embodiment the oxygenate is obtained through fermentation of biomaterials. For example by a process as described in DE-A-10043644.

The preferred molar ratio of oxygenate to olefin in the olefinic co-feed depends on the specific oxygenate used and the number of reactive oxygen-bonded alkyl groups therein. Preferably the molar ratio of oxygenate to olefin lies in the range of 10:1 to 1:10, more preferably in the range of 5:1 to 1:5 and still more preferably in the range of 2:1 to 1:2.

In a preferred embodiment wherein the oxygenate comprises only one oxygen-bonded alkyl group, such as for example methanol or ethanol, the molar ratio preferably lies in the range from 5:1 to 1:5 and more preferably in the range of 2:1 to 1:2. Most preferably the molar ratio in such a case is about 1:1.

In another preferred embodiment wherein the oxygenate comprises two oxygen-bonded alkyl groups, such as for example dimethylether, the molar ratio preferably lies in the range from 5:2 to 1:10 and more preferably in the range of 1:1 to 1:4. Most preferably the molar ratio in such a case is about 1:2.

Step a) of the process is carried out in presence of a one-dimensional zeolite having 10-membered ring channels. These are understood to be zeolites having only 10-membered ring channels in one direction which are not intersected by other 8, 10 or 12-membered ring channels from another direction.

Preferably, the zeolite is selected from the group of TON-type (for example ZSM-22), MTT-type (for example ZSM-23), STF-type (for example SSZ-35), SFF-type (for example SSZ-44)and EU-2-type/ZSM-48 zeolites or mixtures thereof.

MTT-type catalysts are more particularly described in e.g. U.S. Pat. No. 4,076,842. For purposes of the present invention, MTT is considered to include its isotypes, e.g., ZSM-23, EU-13, ISI-4 and KZ-1.

TON-type zeolites are more particularly described in e.g. U.S. Pat. No. 4,556,477. For purposes of the present invention, TON is considered to include its isotypes, e.g., ZSM-22, Theta-1, ISI-1, KZ-2 and NU-10.

EU-2-type zeolites are more particularly described in e.g. U.S. Pat. No. 4,397,827. For purposes of the present invention, EU-2 is considered to include its isotypes, e.g., ZSM-48.

In a further preferred embodiment a zeolite of the MTT-type, such as ZSM-23, or a TON-type, such as ZSM-22, or a mixture thereof is used.

Although other zeolites than the one-dimensional zeolite having 10-membered ring channels may be present, the process of the invention is preferably carried out in the presence of only one or more one-dimensional zeolites having 10-membered ring channels. More preferably the process is carried out in the presence of just one type of one-dimensional zeolites having 10-membered ring channels.

Preferably, a zeolite in the hydrogen form is used, e.g., HZSM-22, HZSM-23, HZSM-35 and HZSM-48. Preferably at least 50% w/w, more preferably at least 90% w/w, still more preferably at least 95% w/w and most preferably 100% of the total amount of zeolite used is zeolite in the hydrogen form. When the zeolites are prepared in the presence of organic cations the zeolite may be activated by heating in an inert or oxidative atmosphere to remove organic cations, for example, by heating at a temperature over 500° C. for 1 hour or more. The hydrogen form can then be obtained by an ion exchange procedure with ammonium salts followed by another heat treatment, for example in an inert or oxidative atmosphere at a temperature over 500° C. for 1 hour or more. The latter zeolites are also referred to as being in the ammonium form.

Preferably the zeolite has a silica to alumina ratio (SAR) in the range from 1 to 500. Preferably the zeolite has a SAR in the range from 10 to 200.

The zeolite can be used as such or in combination with a so-called binder material. When used in the reaction, the zeolite as such or the zeolite in combination with a binder material, are hereafter also referred to as zeolite catalyst.

It is desirable to provide a catalyst having good crush strength, because in an industrial environment the catalyst is often subjected to rough handling, which tends to break down the catalyst into powder-like material. The latter causes problems in the processing. Preferably the zeolite is therefore incorporated in a binder material. Examples of suitable binder materials include active and inactive materials and synthetic or naturally occurring zeolites as well as inorganic materials such as clays, silica, alumina, silica-alumina, titania, zirconia and aluminosilicate. For present purposes, inactive materials of a low acidity, such as silica, are preferred because they may prevent unwanted side reactions which may take place in case a more acidic material, such as alumina or silica-alumina is used. Preferably the catalyst used in the process of the present invention comprises, in addition to the zeolite, 2 to 90 wt %, preferably 10 to 85 wt % of a binder material.

The process of the present invention can be carried out in a batch, continuous, semi-batch or semi-continuous manner. Preferably the process of the present invention is carried out in a continuous manner.

If the process is carried out in a continuous manner, the process may be started up by using olefins obtained from an external source for the olefinic co-feed in step a). Such olefins may for example be obtained from a steam cracker, a catalytic cracker, alkane dehydrogenation (e.g. propane or butane dehydrogenation). Further, such olefins can be bought from the market.

In a special embodiment the olefins for such start-up are obtained from a previous process that converted oxygenates, with or without olefinic co-feed, to olefins. Such a previous process may have been located at a different location or it may have been carried out at an earlier point in time.

In another embodiment an additional catalyst may be used as initiator. After the start-up phase such an initiating catalyst can be removed. Suitable catalysts for this initiating purpose include for example MFI-type catalysts and SAPO-type catalysts.

The reactor used in step a) may be any reactor known to the skilled person and may for example contain a fixed bed, moving bed, fluidized bed and the like.

Conventional catalyst regeneration techniques can be employed. The one-dimensional zeolite having 10 membered ring channels used in the process of the present invention can have any shape known to the skilled person to be suitable for this purpose, for it can be present in the form of tablets, rings, extrudates, etc. Extruded catalysts can be applied in various shapes, such as, cylinders and trilobes. If desired, spent zeolite can be regenerated and recycled to the process of the invention.

Step a) of the process can be carried out over a wide range of temperatures and pressures. Suitably, however, the oxygenate feed and olefinic co-feed are contacted with the zeolite at a temperature in the range from 200° C. to 550° C., preferably from 200° C. to 500° C., more preferably in the range from 250° C. to 450° C. and at an absolute pressure in the range from 1 to 5 bar, more preferably in the range from 1 to 3 bar.

Preferably the oxygenate feed and olefinic co-feed are fed to step a) of the process according to the invention as a vapour, preferably diluted with a diluent gas. Preferably such a diluent gas is an inert gas, such as for example nitrogen or argon. For example, the oxygenate feed and/or olefinic co-feed can be diluted with steam, for example in the range from 0.01 to 10 kg steam per kg feed.

In a further preferred embodiment small amounts of water are added to step a) in order to improve the stability of the catalyst by reducing coke formation.

In step b) of the process according to the invention the olefinic reaction mixture of step a) is separated into at least a first olefinic product fraction and a second olefinic fraction containing C₄ olefins. In step c) at least part of the second olefinic fraction obtained in step b) is recycled to step a) as olefinic co-feed.

Only part of the second olefinic fraction or the complete second olefinic fraction may be recycled to step a). In a preferred embodiment, the second olefinic fraction is separated into two or more further fractions and only part of the second olefinic fraction is recycled to step a).

The separations can be carried out by any method known to the skilled person in the art to be suitable for this purpose, for example by vapour-liquid separation (e.g. flashing), distillation, extraction, membrane separation or a combination of such methods. Preferably the separations are carried out by means of distillation.

In one embodiment, where the purpose of the process is to prepare mainly propene, the olefinic reaction mixture obtained in step a) is preferably separated into at least one olefinic product fraction containing ethene and/or propene and one or more further olefinic fraction containing olefins having 4 or more carbon atoms (i.e. C₄+ olefins), such as butenes, pentenes, hexenes and heptenes, which further olefinic fraction is at least partly recycled.

In a still further embodiment, where the purpose of the process is to prepare ethene, propene, pentene and/or hexene, the olefinic reaction mixture obtained in step a) is preferably separated into a first olefinic product fraction containing ethene and/or propene, a second olefinic product fraction containing pentenes and/or hexenes and a third olefinic fraction containing only olefins having 4 carbon atoms, which third olefinic fraction is at least partly recycled.

### EXAMPLE

In this example a process with a recycle of olefins with more than 4 carbon atoms is simulated. In the example 2-methyl-2-butene (2M2B) and dimethylether (DME) were reacted in a molar feed ratio 2M2B:DME of 2:1 over MFI (comparative) and MTT-type (according to the invention) zeolites of various silica-to-alumina ratios. The silica-to-alumina ratios were respectively 47, 79 and 107 for the MTT-type zeolite. The silica-to-alumina ratio was 280 for the MFI-type zeolite. A sample of zeolite powder was pressed into tablets and the tablets were broken into pieces and sieved. For catalytic testing, the sieve fraction of 30-80 mesh has been used. A quartz reactor tube of 3 mm internal diameter was loaded with 200 mg of sieve fraction. Prior to reaction, the fresh catalyst in its ammonium-form was treated with flowing argon at 550° C. for 1 hour. Next, the catalyst was cooled in argon to the reaction temperature and a mixture consisting of 2.2 vol. % 2M2B, 1.1 vol. % DME and 1 vol. % of water in argon was passed over the catalyst at atmospheric pressure (1 bar) at a flow rate of 50 ml/minute. Periodically, the effluent from the reactor was analyzed by gas chromatography (GC) to determine the product composition. The composition has been calculated on a weight basis. The selectivity has been defined by the division of the mass of product i by the sum of the masses of all products.

The following table (Table 1) lists reaction parameters together with the compositional data, as determined by GC:

TABLE 1

| Zeolite | MFI 280 | MTT 47 | MTT 79 | MTT 107 |
|---|---|---|---|---|
| Time on stream, hours | ~120 | ~120 | ~120 | ~120 |
| Temperature ° C. | 450° C. | 450° C. | 450° C. | 450° C. |
| 2M2B conversion, % | 87 | 67 | 62 | 60 |
| DME conversion, % | 93 | ~100 | ~100 | ~100 |
| Ethylene, wt. %/ | 6/8 | 4.5/7 | 2/3 | 1/2 |
| selectivity, % | | | | |
| Propylene, wt. %/ | 41/49 | 42/66 | 31/54 | 18/32 |
| selectivity, % | | | | |
| Butene isomers, wt. %/ | 30/35 | 14/21 | 12/21 | 10/19 |
| selectivity, % | | | | |
| Hexene isomers, wt. %/ | 5/6 | 3/5 | 13/23 | 27/47 |
| selectivity, % | | | | |
| Heptene isomers, wt. %/ | 2/3 | 1/2 | 1/2 | 3/5 |
| selectivity, % | | | | |

The results show that a high selectivity for propene can be obtained compared to the use of an MFI catalyst. Alternatively, a high selectivity to higher olefins, such as hexane is also achievable.

### COMPARATIVE EXAMPLE A

In this example a mixture of 1-butene and 2-methyl-2-butene was reacted over an MTT-type zeolite. The silica-to-alumina ratio of MTT-type zeolite was 48. A sample of zeolite powder was pressed into tablets and the tablets were broken into pieces and sieved. For catalytic testing, the sieve fraction of 40-60 mesh has been used. The fresh catalyst in its ammonium-form was first treated in air at 600° C. for 4 hours. A quartz reactor tube of 3 mm internal diameter was loaded with 50 mg of catalyst. The reactor was heated in argon to the reaction temperature and a mixture consisting of 2.1 vol. % 1-butene, 2.2 vol % 2-methyl-2-butene and 2 vol. % of water in argon was passed over the catalyst at atmospheric pressure (1 bar) at a flow rates of 100 ml/min. Periodically, the effluent from the reactor was analyzed by gas chromatography (GC) to determine the product composition. The selectivity has been defined by the division of the mass of product i by the sum of the masses of all products. The following table (Table 2) lists reaction parameters together with the compositional data, as determined by GC:

TABLE 2

| Zeolite | MTT |
|---|---|
| Temperature, ° C. | 525° C. |
| 1-butene conversion, % | 6 |
| 2-methyl-2-butene | 64 |
| conversion, % | |
| Ethylene, | 39 |
| selectivity. % | |
| Propylene, | 59 |
| selectivity. % | |
| Hexene isomers, | 1.4 |
| selectivity. % | |
| heptene isomers, | 0.4 |
| selectivity. % | |

The comparative example shows that the selectivity to undesired ethene is enhanced when the conversion of the olefinic feed is carried out in the absence of an oxygenate.

### COMPARATIVE EXAMPLE B

In order to simulate the process without a recycle, methanol (i.e. in the absence of an olefinic co-feed) was reacted over a MTT zeolite with a silica-to-alumina ratio of 48. The reactor was heated in argon to the reaction temperature and a mixture consisting of 8 vol. % methanol in argon was passed over the catalyst at atmospheric pressure at a flow rates of 100 ml/min. Gas hourly space velocity (GHSV) is 60,000, based on total gas flow. Weight hourly space velocities (WHSV) is 6.9 gram methanol/gram catalyst/hr, based on methanol mass flow. The effluent from the reactor was analyzed by mass spectrometry to determine the product composition. The results are recorded in Table 3.

TABLE 3
Product composition of reactor effluent

| Catalyst | MTT-type |
|---|---|
| GHSV, ml/gram/hr | 60,000 |
| Temperature ° C. | 500° C. |
| Time on stream, hr | 1 |
| Methanol conversion, % | 75 |
| Methanol conc., vol. % | 2.0 |
| DME conc., vol. % | 3.0 |
| Total olefin conc., vol. % | <0.2 |

As can be seen from the above the process without any olefinic co-feed results in a low conversion to olefins. This confirms the teaching of EP-A 0485145.

### EXAMPLE 2 AND COMPARATIVE EXAMPLE C

A plug flow, isothermal reactor model is constructed from the measured alkylation and cracking kinetics of a TON-type zeolite catalyst having a silica to alumina ratio of 100 using Aspen Custom Modeler (ACM). Table 4 shows the feed and product molar flow rates for a single-pass reactor using a feed of di-methyl ether (DME) and iso-butene operated at 1 bar, 415° C. and containing 50 tonnes of the zeolite catalyst.

TABLE 4
Single-pass reactor

| | Feed Stream | Product Stream |
|---|---|---|
| Component | (kmol/hour) | (kmol/hour) |
| C₂⁼ | 0 | 164 |
| C₃⁼ | 0 | 2118 |
| C₄⁼ | 1200 | 1539 |
| C₅₊⁼ | 0 | 23 |
| MeOH | 0 | 1 |
| DME | 4077 | 0 |
| H₂O | 0 | 4076 |

Table 5 shows the feed, product and recycle streams for the same reactor operated with a feed of DME without a co-fed olefin but where a recycle stream is applied consisting of all C₄ and higher olefins as well as any unreacted DME and methanol that appear at the reactor outlet. The total feed consists of the feed stream and the recycle stream. The total product consists of the product stream and the recycle stream. As can be seen in table 5, ethene and/or propene can be prepared from oxygenates in just one step with just one zeolite with a sufficient conversion rate, whilst an especially high propene yield is obtained.

TABLE 5
Reactor with recycle

| | Feed Stream | Product Stream | Recycle Stream |
|---|---|---|---|
| Component | (kmol/hour) | (kmol/hour) | (kmol/hour) |
| C₂= | 0 | 227 | 0 |
| C₃= | 0 | 2567 | 0 |
| C₄= | 0 | 0 | 1842 |
| C₅+⁼ | 0 | 0 | 3 |
| MeOH | 0 | 0 | 0 |
| DME | 4077 | 0 | 0 |
| H₂O | 0 | 4077 | 0 |

1. A process for the preparation of an olefin comprising
a) reacting a combination of an oxygenate feed selected from the group consisting of alcohols and ethers, and an olefinic co-feed in a reactor in the presence of a one-dimensional zeolite having 10-membered ring channels, wherein the molar ratio of oxygenate to olefin lies in the range of 10:1 to 1:10, to prepare an olefinic reaction mixture comprising ethene and/or propene;
b) separating the olefinic reaction mixture into at least a first olefinic product fraction and a second olefinic fraction containing olefins with 4 or more carbon atoms; and
c) recycling at least part of the second olefinic fraction obtained in step b) to step a) as olefinic co-feed.

2. The process according to claim 1, wherein the oxygenate is methanol or dimethylether.

3. The process according to claim 1, wherein the zeolite is a zeolite of the MTT-type or the TON-type.

4. The process according to claim 1, wherein the olefinic reaction mixture obtained in step a) is separated into at least one olefinic product fraction containing ethene and/or propene and one or more further olefinic fraction containing olefins having 4 or more carbon atoms, which further olefinic fraction is at least partly recycled.

5. The process according to claim 1, wherein the olefinic reaction mixture obtained in step a) is separated into a first olefinic product fraction containing ethene and/or propene, a second olefinic product fraction containing pentenes and/or hexenes and a third olefinic fraction containing olefins having 4 carbon atoms, which third olefinic fraction is at least partly recycled.

6. The process according to claim 1, wherein the process is started up by using olefins obtained from an external source for the olefinic co-feed in step a).

7. The process according to claim 1, wherein an additional catalyst is used as initiator.

8. The process according to claim 7, wherein the additional catalyst used as initiator is a MFI-type zeolite or a SAPO-type catalyst.

9. The process according to claim 1, wherein the zeolite has a silica to alumina ratio in the range of from 1 to 500.

10. The process according to claim 1, wherein the olefinic co-feed in step a) comprises at least 50 wt % of recycled olefins from step c).

11. The process according to claim 10, wherein the olefinic co-feed in step a) comprises from 90 to 100% of recycled olefins from step c).

12. The process according to claim 1, wherein the olefinic co-feed comprises more than 50 wt %, of olefin(s).

13. The process according to claim 12, wherein the olefinic co-feed comprises more than 80 wt %, of olefin(s).
